(19) United States
(12) Patent Application Publication
(10) Pub. No.: US 2016/0374905 A1
(43) Pub. Date: Dec. 29, 2016

(54) MEDIUM FOR A DENTAL STRUCTURE AND A METHOD OF PRODUCING A MEDIUM FOR A DENTAL STRUCTURE

(71) Applicant: City University of Hong Kong, Kowloon (HK)

(72) Inventors: Xinrui Niu, Kowloon (HK); Yonglong Hu, Yau Ma Tei (HK); Bing Chen, New Territories (HK); Jian Lu, Kowloon (HK)

(21) Appl. No.: 14/751,575

(22) Filed: Jun. 26, 2015

(51) Int. Cl. A61K 6/083; A61K 6/087; A61K 6/00

(52) U.S. Cl. CPC A61K 6/0835; A61K 6/0002; A61K 6/0073; A61K 6/087

(57) ABSTRACT

A medium for a dental structure and a method of producing a medium for a dental structure includes: a polymeric composite arranged to contact with at least one dental component of a dental structure, wherein the polymeric composite further comprises a plurality of inorganic fillers dispersed therein; and wherein the polymeric composite is at least partially translucent after being cured.

### TECHNICAL FIELD

The present invention relates to a method of producing a medium for a dental structure and a medium for a dental structure, and particularly, although not exclusively, to a translucent dental cement and the corresponding fabrication method of the dental cement.

### BACKGROUND

Dental caries, also known as tooth decay, is a widespread phenomenon and a major public health problem affecting most of the people in many countries. It is believed that caries prevalence among children increased in many developed and developing countries.

Untreated tooth decay will cause pain, form dental abscess, and lead to severe local and systemic infections. There are also some evidences indicated that the dental caries would cause heart disease. Blood vessels would be damaged by oral bacteria. Oral bacteria would also cause blood clots by releasing toxins that resemble proteins found in artery walls or the bloodstream. The response of immune system to these toxins could do harm to vessel walls or clot blood more easily. The inflammation in the mouth would have possibility to revs up inflammation throughout the arteries which can lead to heart attack and stroke. Therefore dental caries has to be treated to prevent further health issues.

To restore a decayed tooth or a damaged tooth, the affected dental structure may be restored by applying restoration dental structures to the damaged tooth or teeth. Dental fillings or dental cements may be used to form a portion or even the whole restoration dental structure to replace the damaged portion of the tooth or teeth.

### SUMMARY OF THE INVENTION

In accordance with a first aspect of the present invention, there is provided a medium for a dental structure comprising: a polymeric composite arranged to contact with at least one dental component of a dental structure, wherein the polymeric composite further comprises a plurality of inorganic fillers dispersed therein.

In an embodiment of the first aspect, the polymeric composite is at least partially translucent after being cured.

In an embodiment of the first aspect, the plurality of inorganic fillers is treated in a surface treatment such that a cluster size of the plurality of inorganic fillers is minimized.

In an embodiment of the first aspect, the plurality of inorganic fillers is treated with a surface modifier such that the surface chemistries of the fillers are modified.

In an embodiment of the first aspect, the surface modifier includes at least one of a silane coupling agent, a borate coupling agent, an aluminate coupling agent, a zirconate coupling agent, a titanate coupling agent, a surfactant, a glycidyl compounds, a long chain alkyl acids and an amphiphilic macromolecular compounds.

In an embodiment of the first aspect, the polymeric composite comprises a polymer resin; wherein the polymer resin is at least partially translucent after being cured.

In an embodiment of the first aspect, the polymer resin includes at least one of bis-GMA, TEGDMA, epoxy, and a resin-based polymer with light and/or thermal curable functional groups.

In an embodiment of the first aspect, the plurality of inorganic fillers include at least one of an oxide compound, a carbide compound, a nitride compound, a phosphide compound, a carbonate compound, a carboxylate compound, a phosphate compound, a sulfate compound, a silicate compound, a titanate compound, a zirconate compound, an aluminate compound, a stannate compound, a plumbate compound, a clay containing compound and a glass-containing compound.

In an embodiment of the first aspect, each of the plurality of inorganic fillers includes a dimension in a range from 2 nm to 50 μm.

In an embodiment of the first aspect, the plurality of inorganic fillers includes a shape of at least one of a sphere, a whisker, a platelet, a tube, a fiber, a rod, a sheet, a flake and an irregular shape.

In an embodiment of the first aspect, the polymeric composite comprises a range of weight % of 0-65% of the inorganic fillers.

In an embodiment of the first aspect, the polymeric composite is chemically stable.

In an embodiment of the first aspect, the polymeric composite is arranged to form a component of the dental structure.

In an embodiment of the first aspect, the polymeric composite is arranged to retain a provisional restoration dental structure to the dental structure.

In an embodiment of the first aspect, the medium is dental cement after being cured.

In accordance with a second aspect of the present invention, there is provided a method for producing a medium for a dental structure in accordance with the first aspect, comprising the step of mixing the plurality of inorganic fillers with the polymer resins to form the polymeric composite.

In an embodiment of the second aspect, further comprising the step of:
depositing the polymeric composite to the dental structure; and
curing the polymeric composite.

In an embodiment of the second aspect, further comprising the step of treating the plurality of inorganic fillers in a surface treatment prior to the step of mixing the plurality of inorganic fillers with the polymer resins to form the polymeric composite, such that a cluster size of the plurality of inorganic fillers is minimized.

In an embodiment of the second aspect, the step of treating the plurality of inorganic fillers in a surface treatment comprising the step of mixing a surface modifier with the plurality of inorganic fillers to obtain a plurality of treated inorganic fillers.

In an embodiment of the second aspect, wherein the plurality of treated inorganic fillers comprises a range of weight % of 0-10% of the surface modifier.

In an embodiment of the second aspect, a surface modifier solvent is mixed with the surface modifier and the plurality of inorganic fillers; and wherein the solvent includes at least one of deionized water, methanol, ethanol, propanol, butanol, ethylene glycol, cyclanes, acetone and butanone.

In an embodiment of the second aspect, further comprising the step of purifying the plurality of treated inorganic fillers with a purifying solvent.

In an embodiment of the second aspect, the purifying solvent includes at least one of deionized water and ethanol.

In an embodiment of the second aspect, the step of purifying the plurality of treated inorganic fillers with the purifying solvent is repeated 0-10 times.

In an embodiment of the second aspect, further comprising the step of drying the plurality of treated inorganic fillers with an oven and/or a freeze dryer.

In an embodiment of the second aspect, the plurality of treated inorganic fillers with the polymer resins are mixed by at least one of a high speed mixer, ultra-sonicator and a three roll miller.

### BRIEF DESCRIPTION OF THE DRAWINGS

Embodiments of the present invention will now be described, by way of example, with reference to the accompanying drawings in which:

FIG. 1A is a cross-sectional illustration of a dental structure with a medium for a dental structure in accordance with an embodiment of the present invention;

FIG. 1B is a cross-sectional illustration of a dental structure with a medium for a dental structure in accordance with another embodiment of the present invention;

FIG. 2 is an illustration of examples of the shapes of the inorganic fillers included in the medium of FIG. 1A;

FIG. 3A is a plot showing the relationship between the translucency, the filler loading ratio and the Young's modulus of the medium for a dental structure in accordance with an embodiment of the present invention;

FIG. 3B is a plot showing the relationship between the translucency, the size of fillers and the Young's modulus of the medium for a dental structure in accordance with an embodiment of the present invention;

FIG. 4A is a microscopic image of inorganic fillers in forms of aggregates or large clusters;

FIG. 4B is a microscopic image of inorganic fillers in forms of minimized clusters;

FIG. 4C is an illustration of an isolated inorganic filler particle modified by a surface modifier;

FIG. 5A is a process flow diagram showing the method of producing the medium of FIG. 1A, the detail steps of the surface treatment process are shown;

FIG. 5B is a process flow diagram showing the method of producing the medium of FIG. 1A, the detail steps of the purifying process are shown;

FIG. 5C is a process flow diagram showing the method of producing the medium of FIG. 1A, the detail steps of the drying process are shown;

FIG. 5D is a process flow diagram showing the method of producing the medium of FIG. 1A, the detail steps of the mixing process are shown;

FIG. 5E is a process flow diagram showing the method of producing the medium of FIG. 1A, the detail steps of the curing process are shown;

FIG. 6A is a microscopic image of unmodified 20% ZrO₂/Epoxy cement;

FIG. 6B is a microscopic image of modified 20% ZrO₂/Epoxy cement;

FIG. 7 is a plot showing the comparison of the mechanical properties of the unmodified and modified ZrO₂/Epoxy cement of different loading ratio;

FIG. 8 is a plot showing the transparency of several examples of dental cements with different fillers.

### DETAILED DESCRIPTION OF THE PREFERRED EMBODIMENT

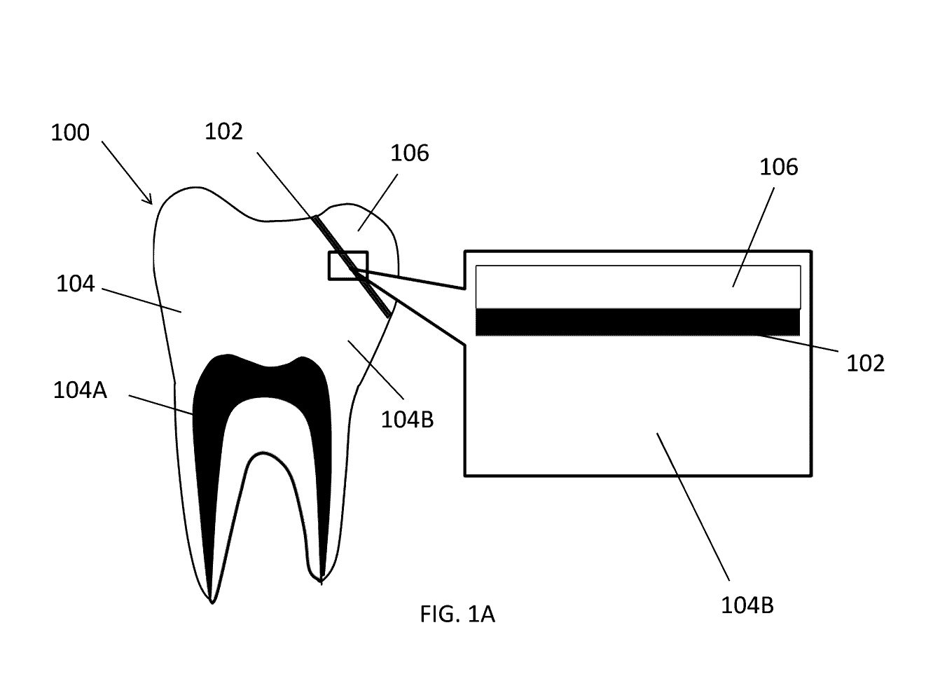

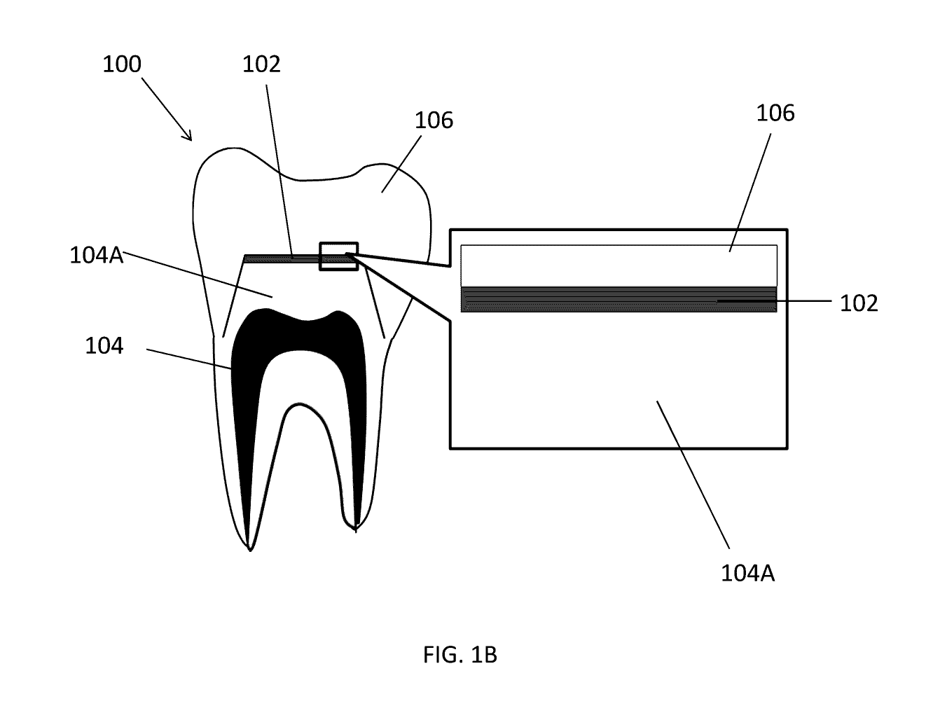

With reference to FIGS. 1A and 1B, there is provided an example embodiment of a medium 102 for a dental structure 100 comprising: a polymeric composite arranged to contact with at least one dental component of a dental structure 100, wherein the polymeric composite further comprises a plurality of inorganic fillers dispersed therein; and wherein the polymeric composite is at least partially translucent.

In this embodiment, the medium 102 is arranged to contact with a tooth 104 and a prosthesis 106 (such as provisional crowns and bridges) in a dental structure 100. In this example, the medium 102 is used as a dental cement for retaining a provisional restoration dental structure 106 (such as metallic, plastic or ceramic restorations) to the dentin 104A or the enamel layer 104B of the tooth 104, as well as protecting the pulp from injury by forming an insulating layer between the restorations 106 and the dentin 104A.

Referring to the examples as shown in FIGS. 1A and 1B, the medium 102 may be used for restorations, such as bonding restorative material 106 to broken enamel 104B for provisional restorations or bonding ceramic crowns 106 to dentin 104A for relatively long-term (or permanent) restorations.

The medium 102 comprises a polymeric composite, preferably a curable polymeric composite which may be freely shaped before the composite is cured, such that the polymeric composite is arranged to fill up any gaps between the restoration dental structure 106 and the tooth 104 and hence enhancing the attachment of the restoration structure 106 or the prosthesis 106 to the tooth structure 104. Preferably, the polymeric composite includes a polymer resin and the polymer resin may include light and/or thermal curable functional group or the resin may be self-cured. For example, the polymer resin includes at least one of the following compositions including but not limited to bis-GMA (Bisphenol A Glycidyl Methacrylate), TEGDMA (triethyleneglycol-dimethacrylate), epoxy, and a resin-based polymer.

In addition, the medium 102 comprises a plurality of inorganic fillers so as to strengthen the medium 102 or the polymeric composite. The inorganic fillers may have generally stronger mechanical properties than the pure polymer resins, which enable the fillers to sustain a higher stress than the polymer resins do. The inorganic fillers may also hinder the deformation of the cured polymeric composite therefore may increase the rigidity of the medium 102. In addition, the inorganic fillers may deviate the propagation of the micro-cracks within the medium 102.

Preferably, the plurality of inorganic fillers include at least one of an oxide compound, a carbide compound, a nitride compound, a phosphide compound, a carbonate compound, a carboxylate compound, a phosphate compound, a sulfate compound, a silicate compound, a titanate compound, a zirconate compound, an aluminate compound, a stannate compound, a plumbate compound, a clay containing compound and a glass-containing compound. Alternatively, the medium 102 may comprise any other types of fillers as known by a person skilled in the art for strengthening the medium 102 for a dental structure 100.

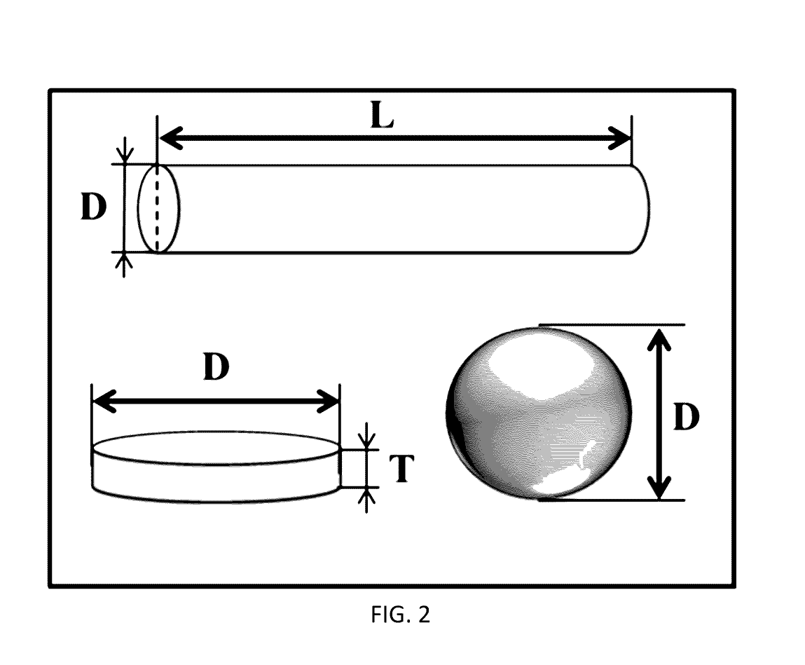

Preferably, each of the plurality of inorganic fillers includes a dimension in a range from 2 nm to 50 μm (more preferably from 5 nm to 30 μm), and a shape selected from one or more of a sphere, a whisker, a platelet, a tube, a fiber, a rod, a sheet, a flake and an irregular shape. The dimensions may be defined as any one of a diameter (D) of a sphere/platelet/rod, a length (L) of a tube/fiber/rod or a thickness (T) of a platelet/sheet/flake of the exemplary shapes as shown in FIG. 2, or any other reasonable definitions of a dimension of shape.

The medium 102 may comprises various compositions of polymeric composite and inorganic fillers optimized for different applications. Preferably, the combination of the polymer resins and the plurality of inorganic fillers comprises a range of weight % of 0-65% (more preferably in a range of weight % of 1-55%) of the inorganic fillers. With different weight %, sizes and shapes of inorganic fillers within the polymeric composite, the physical/mechanical properties and hence the strengthening effect of the inorganic fillers in the medium 102 may be optimized.

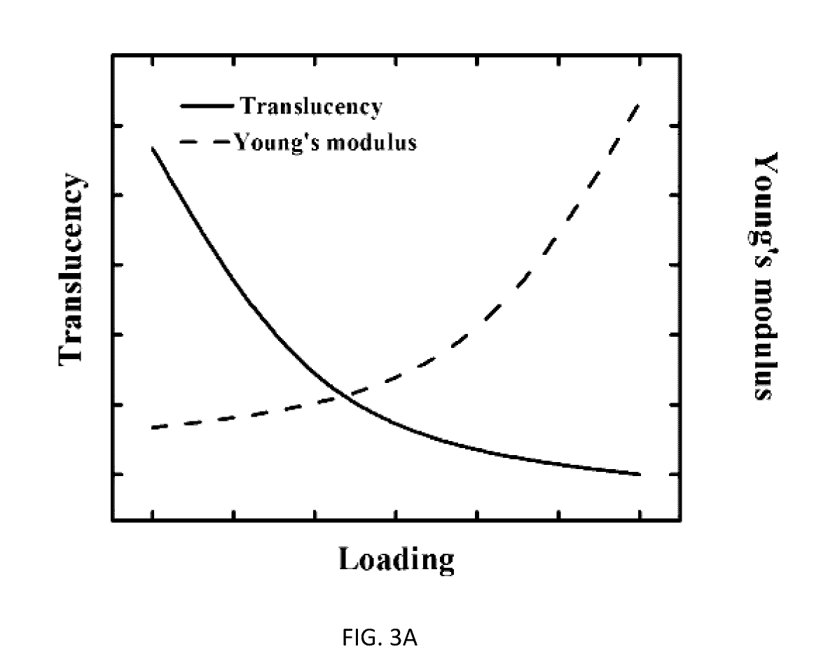

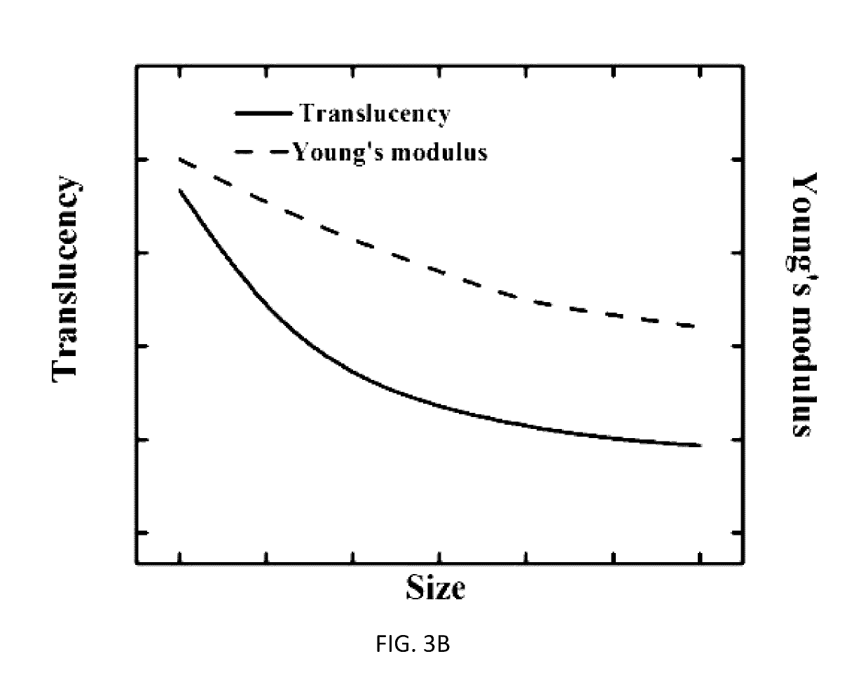

With reference to FIGS. 3A to 3B, the medium 102 which comprises the polymeric composite comprising the resins and the fillers is at least partially translucent or transparent after being cured. The transparency of the medium 102 may be adjusted by including a different composition of polymeric composite with different transparency, or by introducing different amounts/sizes/shapes of inorganic fillers which may affect the scattering, reflection, refraction and/or absorption of light passing through the translucent or transparent polymeric composite. It is also shown in the Figures that the mechanical properties such as the Young's modulus of the medium 102 may be adjusted by varying the loading ratio of the inorganic fillers in the medium 102.

For example, the translucency of the dental cement may be affected by polymeric resins, fillers, and surface modifiers. For polymeric resins, the higher the translucency or even transparency results in greater cement translucency, and the vice versa. For inorganic fillers, the larger the particle sizes, and/or the higher the loadings, and/or the lower the translucency or transparency, the lower the translucency of the cements, and the vice versa. For surface modifiers (used in surface treatment which will be explained in more detail below), those which have no color or lighter colors lead to better cement translucency. Also the modifiers can enhance the dispersibility of fillers to increase the translucency of cements. Besides, the mechanical properties such as strength, Young's modulus and toughness will also be tuned. For example, higher loadings results in higher Young's modulus, and the vice versa.

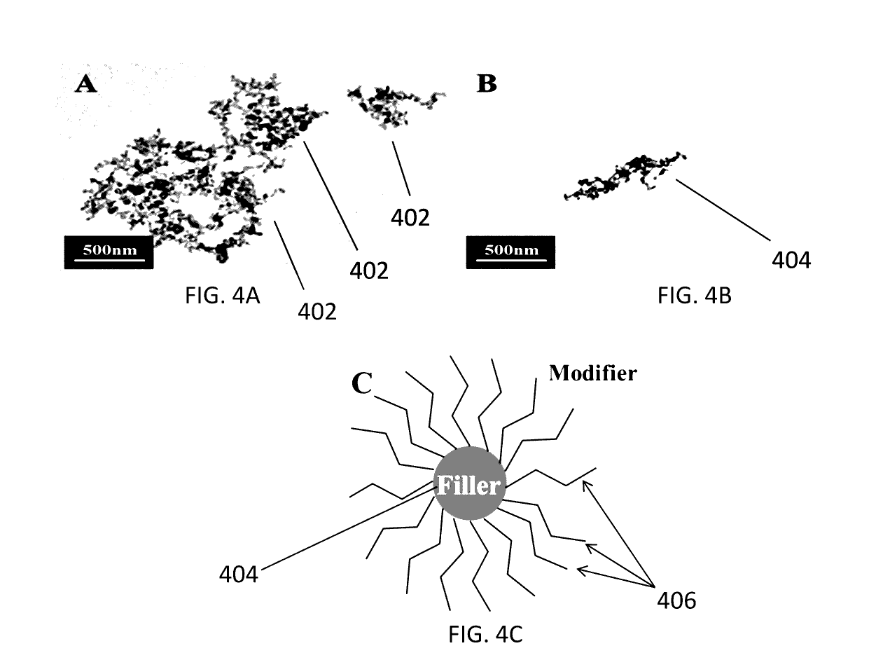

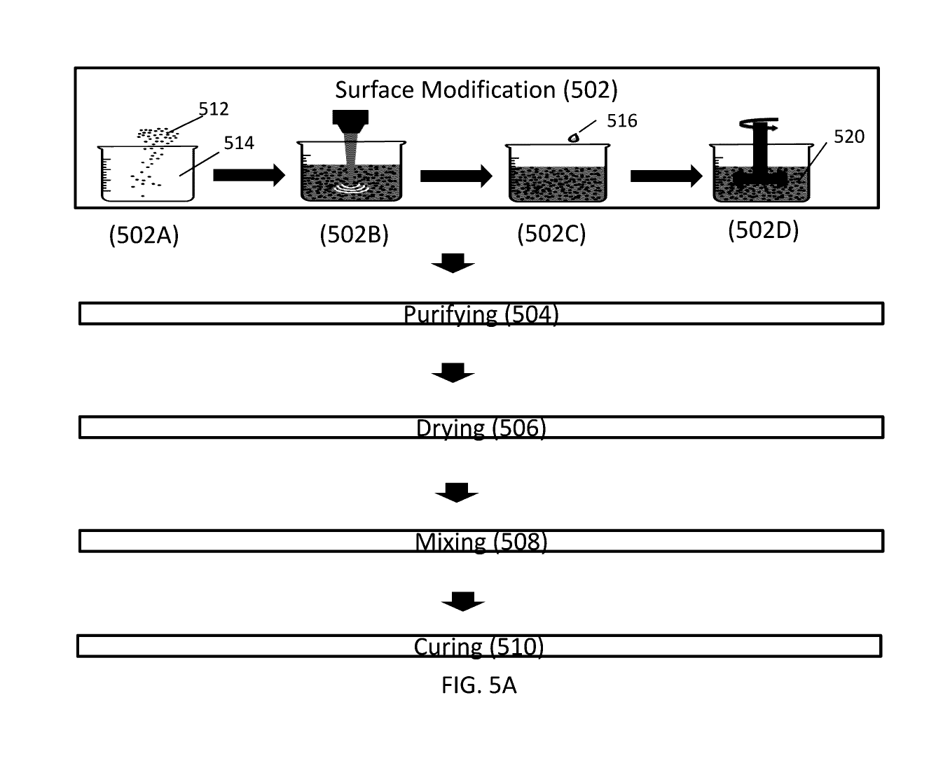

In this example embodiment, the plurality of inorganic fillers is treated in a surface treatment such that a cluster size of the plurality of inorganic fillers is minimized With reference to FIGS. 4A to 4B, it is shown that inorganic fillers with fine filler size like nanoparticles, exist more preferably as aggregates or larger clusters 402. However, inorganic fillers treated in a surface treatment or a surface modification exist as minimized cluster 404 as shown in FIG. 4B. This is due to the steric effect of the surface modifier 406 grafted or covered on the particle surface 404 as illustrated in FIG. 4C.

Advantageously, the surface modifiers change the surface properties of inorganic fillers and enhance the compatibility of fillers with polymer resins. As a result, the inorganic fillers will be more stable in polymer resins and the dispersibility of the inorganic fillers becomes better.

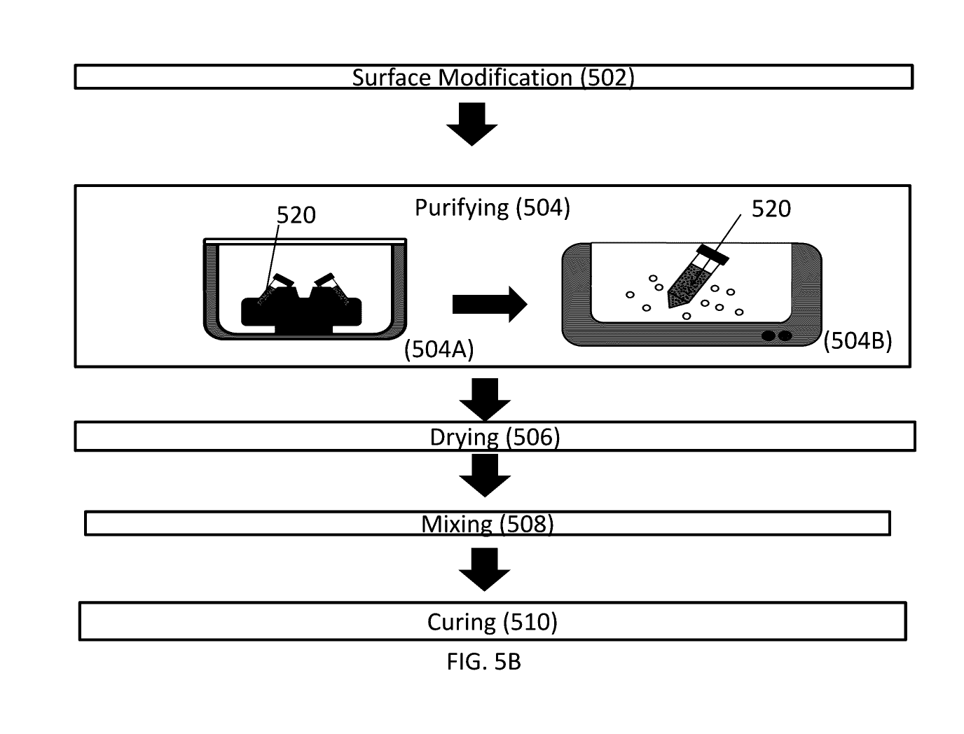

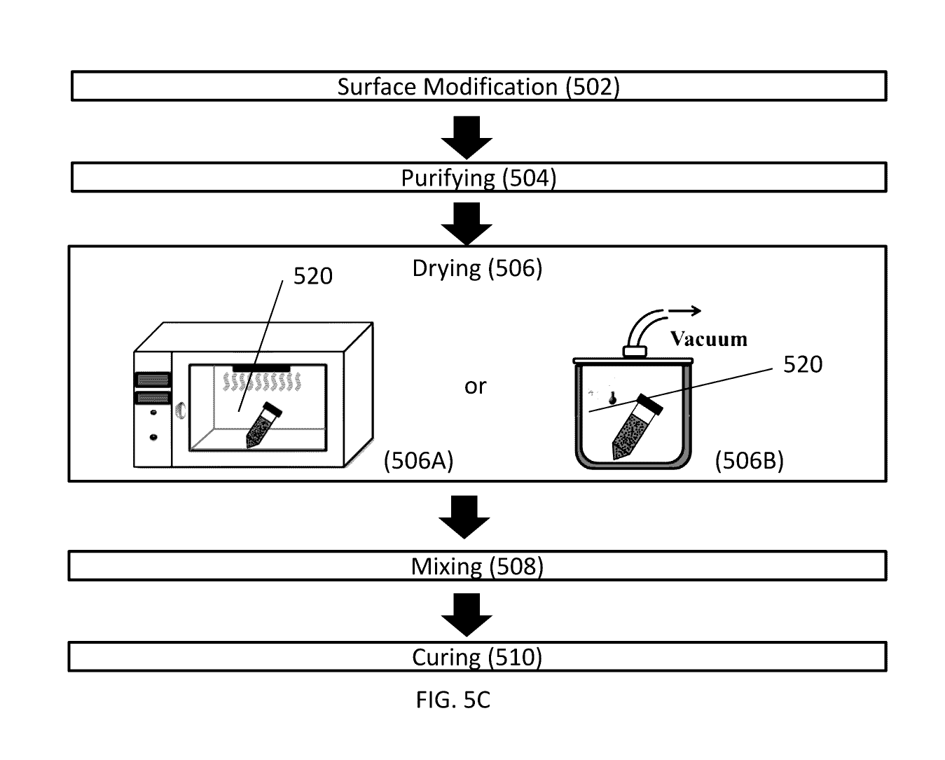

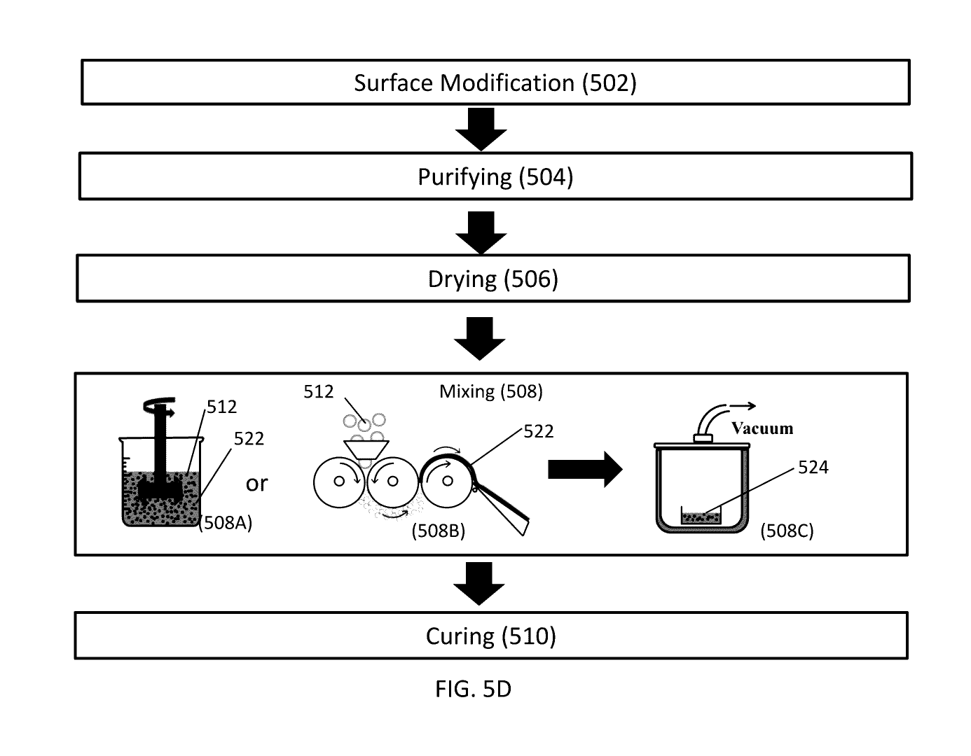

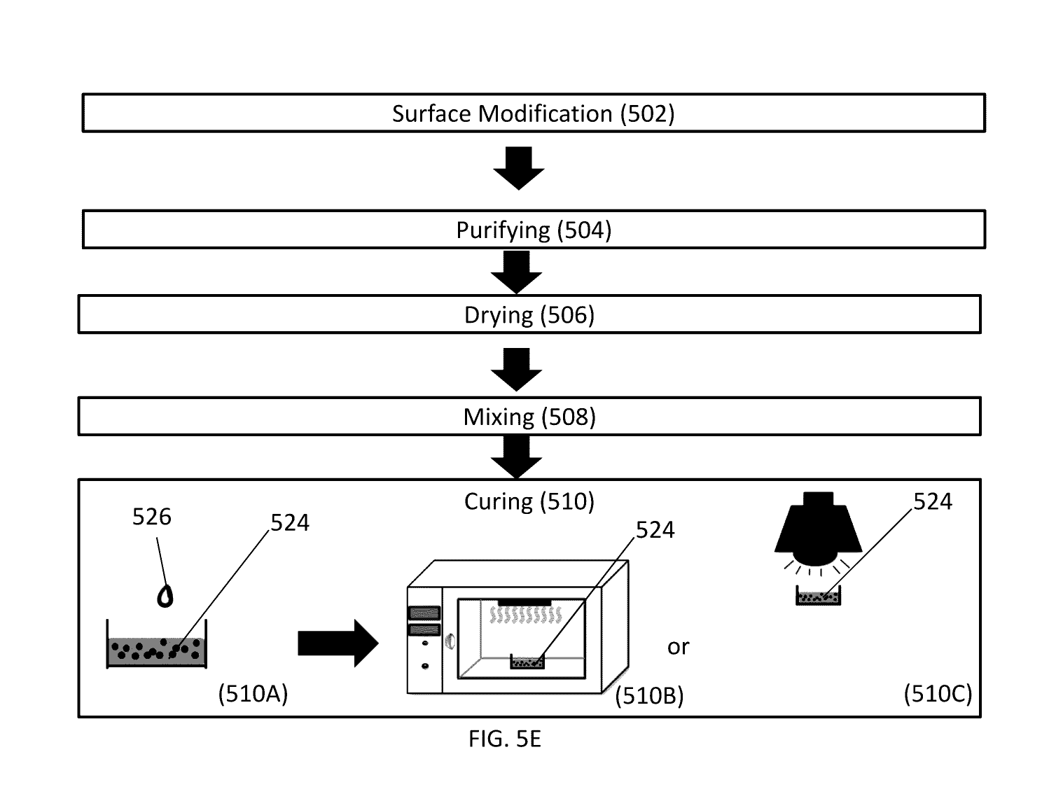

With reference to FIGS. 5A to 5E, there is provided an example embodiment of a method for producing a medium 102 for a dental structure 100, comprising the step of mixing the plurality of inorganic fillers with the polymer resins to form the polymeric composite. This mixture may be further packaged.

Preferably, prior to the step of mixing the fillers and the polymer resins together to form a mixture or the polymeric composite, the inorganic fillers are treated in a surface treatment, such that a cluster size of the inorganic fillers is minimized since the treated inorganic fillers has a better dispersibility than those without surface treatment. These treated inorganic fillers having excellent dispersibility may then dispersed in the polymer resins more evenly and hence enhancing the transparency of the medium 102 after the polymeric composite is cured. In addition, the excellent dispersion of the inorganic fillers in the polymer resins also enhances the chemical bonding between the fillers and the polymer resins.

In an exemplary embodiment of treating the inorganic fillers in a surface treatment, the plurality of inorganic fillers is treated with a surface modifier in Step 502. For example, starting with step 502A, the inorganic filler 512 is mixed with a surface modifier solvent 514 and the mixture is further treated by ultrasonication in step 502B. After that, in the step 502C of treating the plurality of inorganic fillers in a surface treatment, a surface modifier 516 is mixed with the plurality of inorganic fillers 512. Preferably, the mixture of the plurality of inorganic fillers 512 and the surface modifier 516 comprises a range of weight % of 0-10% of the surface modifier 516, and the surface modifier 516 includes at least one of a silane coupling agent, a borate coupling agent, an aluminate coupling agent, a zirconate coupling agent, a titanate coupling agent, a surfactant, a glycidyl compounds, a long chain alkyl acids and an amphiphilic macromolecular compounds. Alternatively, the amount and the type of surface modifier may be selected differently in accordance with the fillers 512 and/or the polymer resins 522 used for producing the medium 102 or the dental cement. Subsequently, in step 502D, the mixture 520 containing the fillers 512, the solvent 514 and the surface modifier 516 may be further stirred by a stirrer or a mixer.

Optionally, the above method for producing a medium 102 for a dental structure 100 further comprises a step of purifying the plurality of inorganic fillers with a purifying solvent, in which the inorganic fillers are further purified in the steps of 504 as shown in the FIG. 5B. In this example, the mixture 520 with the treated fillers 512 and the solvent 514 are centrifuged in step 504A, and then processed by ultrasonication in step 504B, after replace the solvent 514 with purifying solvent selected from at least one of deionized water and ethanol. This purifying process (steps 504A-504B) may be repeated 0-10 times based on the requirement of purity of the treated fillers 512. The mixture 520 is further processed in an oven in step 506A and/or a freeze dryer in step 506B to drive away the purifying solvent so as to obtain dried and purified treated fillers 512 for further process.

Subsequently, the treated fillers 512 may then be mixed with the polymer resins 522 with the help of a mixing apparatus, such as but not limited to a high speed mixer (step 508A), ultra-sonicator and/or a three rolling miller (step 508B). Optionally, a degassing process (step 508C) may be performed to the mixture 524 comprising the polymer resins 522 and the treated fillers 512. The mixture 524 may then be applied to the desired dental structure 100.

Finally, after a curing process (step 510) of the polymeric composite, the polymeric composite is chemically stable. For example, the mixture 524 comprising the polymer resins 522 and the treated fillers 512 may be cured by thermal curing in step 510B and/or light curing in step 510C. Optionally, curing agents 526 may be added to the mixture 524 in step 510A prior to the steps 510B and/or 510C to facilitate the curing process.

This chemically stable medium 102 comprising the polymeric composite or the combination of the polymer resins and the fillers may form a component of the dental structure 100. For example, the medium 102 may be used as dental fillings for restoring a decayed tooth. Alternatively, the medium 102 may be applied as a bonding member between a dental structure 100 (such as a tooth) and a provisional restoration dental structure 106 for retaining the provisional restoration dental structure 106 to the tooth 104.

The above embodiments may be advantageous in that the medium for a dental structure may be used for a variety of dental and/or orthodontic applications, such as luting agents, pulp-protecting agents or cavity-lining material. In an example embodiment, the translucent dental cement may be directly applied at a failure area of a damage dental structure, such that the dental cement may bond the restorative material to crown dentin or dental composites after short time curing.

The advantages of surface treatment for inorganic fillers cover not only the bonding strength enhancement of organic-inorganic interfaces, but also the achievement of more uniformly dispersed inorganic fillers in the medium.

In addition, the physical/mechanical properties and the transparency of the medium may be easily tuned or adjusted by including different amount/type/composition of inorganic fillers for different application. For example, a luting agent for bonding a prosthesis may not necessary to include a high transparency could contains a higher loading ratio of inorganic fillers for enhancing the mechanical properties of the medium. However, for some possible applications that the cured dental cement applied may be visible, a higher transparency of the medium may be preferred to improve the overall appearance of the dental structure.

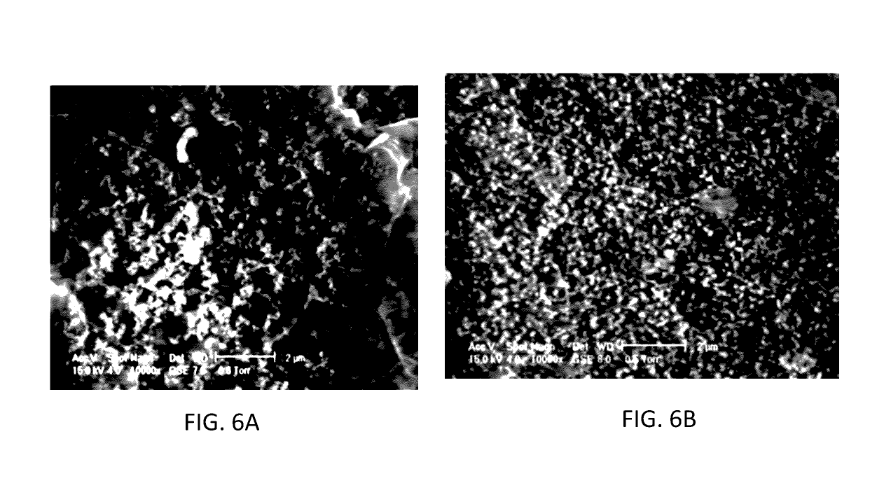

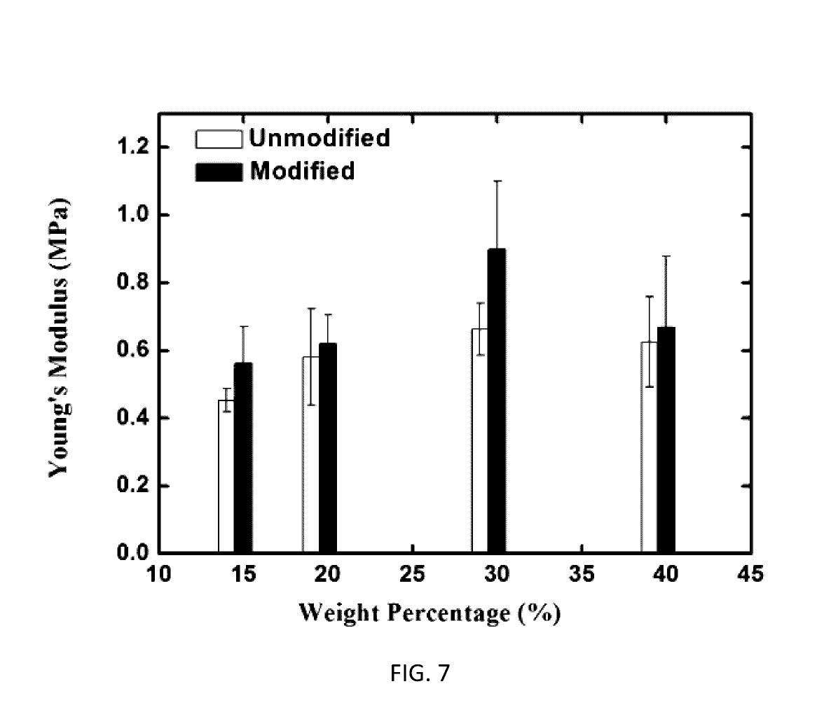

With reference to FIGS. 6A and 6B, there is shown the comparison of a medium for a dental structure in accordance with an embodiment of the present invention. The medium of FIG. 6A contains unmodified 20% ZrO₂/Epoxy and the one of FIG. 6B contains modified 20% ZrO₂/Epoxy. It is obvious that the medium with modified fillers does not contain an aggregate or a cluster as shown in FIG. 6A. In addition, with reference to FIG. 7, it is shown that a medium with modified fillers has a generally better mechanical strength than the one with unmodified fillers.

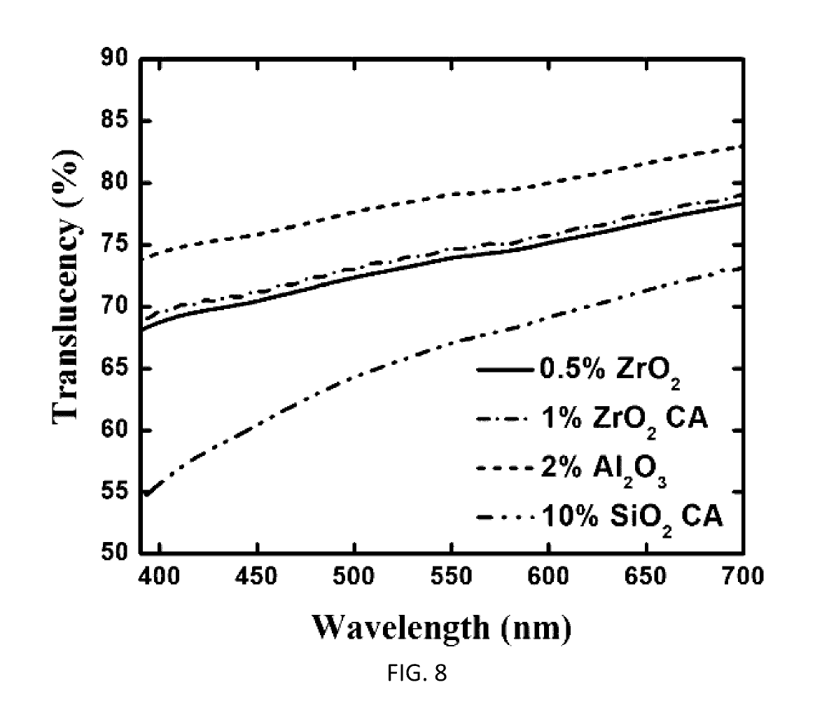

With reference to FIG. 8, there is shown several example embodiments of a translucent dental cement of the present invention. The medium may comprises a measured translucency varies from 55% up to 85% within the visible light spectrum.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A medium for a dental structure comprising: a polymeric composite arranged to contact with at least one dental component of a dental structure, wherein the polymeric composite further comprises a plurality of inorganic fillers dispersed therein; and wherein the polymeric composite is at least partially translucent after being cured.

2. A medium for a dental structure in accordance with claim 1, wherein the plurality of inorganic fillers is treated in a surface treatment such that a cluster size of the plurality of inorganic fillers is minimized.

3. A medium for a dental structure in accordance with claim 2, wherein the plurality of inorganic fillers is treated with a surface modifier such that the surface chemistries of the fillers are modified.

4. A medium for a dental structure in accordance with claim 3, wherein the surface modifier includes at least one of a silane coupling agent, a borate coupling agent, an aluminate coupling agent, a zirconate coupling agent, a titanate coupling agent, a surfactant, a glycidyl compounds, a long chain alkyl acids and an amphiphilic macromolecular compounds.

5. A medium for a dental structure in accordance with claim 1, wherein the polymeric composite comprises a polymer resin; wherein the polymer resin is at least partially translucent after being cured.

6. A medium for a dental structure in accordance with claim 5, wherein the polymer resin includes at least one of bis-GMA, TEGDMA, epoxy, and a resin-based polymer with light and/or thermal curable functional groups.

7. A medium for a dental structure in accordance with claim 1, wherein the plurality of inorganic fillers include at least one of an oxide compound, a carbide compound, a nitride compound, a phosphide compound, a carbonate compound, a carboxylate compound, a phosphate compound, a sulfate compound, a silicate compound, a titanate compound, a zirconate compound, an aluminate compound, a stannate compound, a plumbate compound, a clay containing compound and a glass-containing compound.

8. A medium for a dental structure in accordance with claim 1, wherein each of the plurality of inorganic fillers includes a dimension in a range from 2 nm to 50 μm.

9. A medium for a dental structure in accordance with claim 1, wherein the plurality of inorganic fillers includes a shape of at least one of a sphere, a whisker, a platelet, a tube, a fiber, a rod, a sheet, a flake and an irregular shape.

10. A medium for a dental structure in accordance with claim 1, wherein the polymeric composite comprises a range of weight % of 0-65% of the inorganic fillers.

11. A medium for a dental structure in accordance with claim 1, wherein the polymeric composite is chemically stable.

12. A medium for a dental structure in accordance with claim 1, wherein the polymeric composite is arranged to form a component of the dental structure.

13. A medium for a dental structure in accordance with claim 1, wherein the polymeric composite is arranged to retain a provisional restoration dental structure to the dental structure.

14. A medium for a dental structure in accordance with claim 1, wherein the medium is a dental cement.

15. A method for producing a medium for a dental structure in accordance with claim 1, comprising the step of mixing the plurality of inorganic fillers with the polymer resins to form the polymeric composite.

16. A method for producing a medium in accordance with claim 15, further comprising the step of:
depositing the polymeric composite to the dental structure; and
curing the polymeric composite.

17. A method for producing a medium in accordance with claim 15, further comprising the step of treating the plurality of inorganic fillers in a surface treatment prior to the step of mixing the plurality of inorganic fillers with the polymer resins to form the polymeric composite, such that a cluster size of the plurality of inorganic fillers is minimized.

18. A method for producing a medium in accordance with claim 17, wherein the step of treating the plurality of inorganic fillers in a surface treatment comprising the step of mixing a surface modifier with the plurality of inorganic fillers to obtain a plurality of treated inorganic fillers.

19. A method for producing a medium in accordance with claim 18, wherein the plurality of treated inorganic fillers comprises a range of weight % of 0-10% of the surface modifier.

20. A method for producing a medium in accordance with claim 18, wherein a surface modifier solvent is mixed with the surface modifier and the plurality of inorganic fillers; and wherein the solvent includes at least one of deionized water, methanol, ethanol, propanol, butanol, ethylene glycol, cyclanes, acetone and butanone.

21. A method for producing a medium in accordance with claim 18, further comprising the step of purifying the plurality of treated inorganic fillers with a purifying solvent.

22. A method for producing a medium in accordance with claim 21, wherein the purifying solvent includes at least one of deionized water and ethanol.

23. A method for producing a medium in accordance with claim 21, wherein the step of purifying the plurality of treated inorganic fillers with the purifying solvent is repeated 0-10 times.

24. A method for producing a medium in accordance with claim 21, further comprising the step of drying the plurality of treated inorganic fillers with an oven and/or a freeze dryer.

25. A method for producing a medium in accordance with claim 15, wherein the plurality of treated inorganic fillers and the polymer resins are mixed by at least one of a high speed mixer, ultra-sonicator and a three roll miller.
